(19) United States
(12) Patent Application Publication
(10) Pub. No.: US 2009/0088705 A1
(43) Pub. Date: Apr. 2, 2009

(54) APPLICATION SYSTEM FOR TOPICALLY APPLIED COMPOSITIONS

(76) Inventor: Neil SIRKIN, Mountain Brook, AL (US)

Correspondence Address: WILLIAM J. SAPONE;COLEMAN SUDOL SAPONE P.C., 714 COLORADO AVENUE, BRIDGE PORT, CT, 06605, US

(21) Appl. No.: 11/866,190

(22) Filed: Oct. 2, 2007

(51) Int. Cl. A61F 13/15; A61F 13/84

(52) U.S. Cl. 604/290; 206/438; 604/289

(57) ABSTRACT

The present invention provides a system and method of delivery of topical compositions, such as povidone iodine, anti-itch compositions or anti-fungal compositions, for home use that is effective, yet spreading to non-target areas. A plurality of absorbent pads are located in a container, a fluid containing the composition absorbed onto the pads, the pads being sufficiently absorbent to accept a unit dose of the fluid therein, such that the pads in the container when handled are moist but not soaking, the dose applied with slight pressure when the pad contacts the target area, thereby avoiding spread of the fluid to non-target areas, or dripping onto nearby surfaces.

### TECHNICAL FIELD

This invention relates to a system for applying various compositions to the skin, and more particularly to a system and method for applying compositions to specific target skin areas of the skin, with a reduced susceptibility to contacting other not target areas, and more particularly to applying povidone-iodine to the skin in the home, without causing staining of other skin areas or adjacent surfaces.

### BACKGROUND

The use of absorbent pads to apply various compositions to the skin is known. For example, cotton balls are often used to spread liquids over the skin. Generally, the liquid is poured onto the cotton ball, or the ball is immersed in the liquid, the infused cotton ball they applied to the skin. It is not uncommon for the fluid to spill somewhat, and to spread to not target areas, but in many instances this is an accepted inconvenience.

A similar known application system is the Stridex® medicated pad system, where a jar contains a liquid solution of salicylic acid and alcohol. The pads absorb the solution which is applied to the skin using the pad, which provides an additional abrasive component for cleaning the skin, as these pads are used to treat acne. However, as with the cotton application, it is an accepted inconvenience that some of the liquid may drip off the pad, or spread to areas other than the contact areas.

Povidone-iodine is a known disinfecting composition, often used to prepare skin for incisions, prior to surgery. It is very common for use in hospitals and in other professional medical settings. However, use in the home has encountered problems. In particular, povidone-iodine is a strongly colored fluid, which has a very high capacity for staining virtually anything it comes in contact with. Thus, any stray fluid, on the skin, or dripped on carpet, or countertops can cause long lasting staining.

Many medicinal liquids that need to be applied to the skin often are in a form where spreading to other skin areas, dripping, etc. occur, which is often at a minimum, inconvenient to the user. In many instances, this also wastes a significant portion of the active ingredients, requiring additional purchases to make up for lost product. In some instances, avoiding skin to skin contact to prevent spread of the condition can also be a concern, and so limiting contact application to specific target areas has higher importance. This is a particular concern when applying gels or creams.

### SUMMARY OF THE INVENTION

It is an object of the present invention to provide a system and method of delivery of topical compositions, such as povidone iodine, anti-itch compositions or anti-fungal compositions, for home use that is effective, yet additionally limits the potential for spreading to non-target areas, or in the case of povidone iodine, staining non target areas and other surfaces.

The invention comprises a plurality of absorbent pads, a container for holding the pads, and a fluid located in the container, the pads being sufficiently absorbent to accept a unit dose of the fluid therein, such that the pads in the container when handled are moist but not soaking, the dose applied with slight pressure when the pad contacts the target area, thereby avoiding spread of the fluid to non-target areas, or dripping onto nearby surfaces.

Using the present invention, the composition is retained in the pad when handled, and only dispensed when applied with slight pressure onto the skin. Such a system and method prevents dripping or spreading of the fluid, enabling home use while limiting the potential for staining or damaging adjacent surfaces.

The present invention is distinguishable from the prior medicated pads, as liquid is not likely to drip off or spread to a non-target skin area, avoiding the inconvenience of the prior art.

### DETAILED DESCRIPTION OF THE INVENTION

The inventive system comprises a container, such as a jar which contains from 10 to 100 individual pads arranged in a stack, each pad being preferably round, though they may be square or another shape, and having a surface area of from about 2 to 15 square inches, and a thickness of from about 0.25 to 0.50 mils. The material is chosen from various known absorbent materials, preferably being a non-woven fabric of blended synthetic fibers. One example is a blend of rayon/polyethylene fibers bonded together, though other absorbent materials may be used to produce the absorbent pads. A 75/25 ratio of rayon/polypropylene is particularly preferred.

The container is of conventional design, sized for retaining the specific pads to be used, and may be made of glass, plastic.

The fluid is dosed into the container, and the pads added, the pads absorbing the liquid such that each pad contains approximately a unit dose of the fluid. Typically, the pad itself is about 75 gram Novonette®, a nonwoven fabric material, each pad being capable of absorbing up to about 1.5 grams of the fluid. The method of forming the pads may comprise running a three inch ribbon of waffle Novonette® through a feeding machine and thereafter cutting to shape, and stacking, the stack then placed in the jar either containing the fluid, or with the fluid applied thereafter. It is also contemplated that the pads would be dipped into the solution so as to impregnate the pads before these are placed in the container.

The fluid is preferably provided at from 0.1-15% by weight, and is preferably a solution containing povidone-iodine, at from 1-15%, more preferably the fluid is povidone-iodine, 10%, USP. Other fluids that can be delivered with the inventive system and method are:

Tolnaftate solution, at from 0.1-15% by weight, preferably 1%, USP, antifungal (Tolnaftate is an antifungal medicine used primarily to treat ringworm and tinea infections, such as athlete's foot);

Hydrocortisone solution, at from 0.1-15% by weight, preferably 1%, USP, antipruritic;

Diphenhydramine solution, at from 0.1-15% by weight, preferably 2%, USP, antihistamine;

Clotrimazole solution, at from 0.1-15% by weight, preferably 1%, USP, antifungal (Clotrimazole is another antifungal medicine used primarily to treat ringworm and tinea infections, such as athlete's foot).

Antifungal compositions are particularly suited to the present invention, as the pads prevent direct contact of the hands or fingers with an infected area, to limit spread of the fungus thereto. Of course, the inventive method and system can be used with other fluids that can benefit from the reduced spread of the fluid and/or avoidance of direct contact with the treatment area, and the invention is not limited to those discussed by way of example, as these other fluids can be packaged for home use and then applied in the same way. The invention is thus not limited to these particular fluids/compositions. The following are exemplary formulas.

### Example 1

Fluid Containing Povidone-lodine Solution 10% USP

| COMPONENT | W/W % |
|---|---|
| Povidone-Iodine, USP | 10.000 |
| Glycerin (96%), USP | 0.840 |
| Citric Acid Anhydrous, USP | 0.097 |
| Sodium Phosphate, Dibasic Heptahydrate | 0.129 |
| (7H₂O) | |
| Sodium Hydroxide, NF | 0.161 |
| surfactant (Igepal CO 630) | 0.258 |
| Purified Water, USP | 88.515 |
| Total | 100.00% |

Manufacturing Instructions:
1. To a kettle add the Purified Water
2. While mixing, add: Glycerin; Citric Acid Anhydrous Sodium Phosphate, Dibasic, Heptahydrate Sodium Hydroxide Igepal CO 630
3. Mix until dissolved, then with continued mixing, add: Povidone-lodine, USP

### Example 2

Fluid Containing Tolnaftate Topical Solution Usp, 1%

| Material | % Used |
|---|---|
| Peg 400, Nf (a low molecular weight grade | 99.017 |
| of polyethylene glycol) | |
| Butylated Hydroxytoluene, Nf | 00.089 |
| Tolnaftate, Usp | 00.894 |

Manufacturing Instructions:
1. Put Peg 400, Nf In Kettle, Heat To 40-45° C.
2. With Mixing Add Butylated Hydroxytoluene, Mix Until Dissolved;
3. With Mixing Add Tolnaftate, Usp, Mix Until Dissolved.

### Example 3

Fluid Containing An Anti-itch Composition 2.0% W/V

| Material | % Used |
|---|---|
| Purified Water | 2.66 |
| Tris (Hydroxymethyl) Aminomethan | 0.6 |
| Alcohol Sd-40a | 88.8 |
| Diphenydramine Hcl | 2.42 |
| Povidone, Usp | 2.61 |
| Acetulan(Lanaetex-75) | 2.61 |
| Wheat Germ Glycerides | 0.3 |

Manufacturing Instructions:
1. Put Purified Water And Tris Aminomethan In A Kettle And Mix Until Dissolved
2. Into Another Kettle Add Alcohol And With Mixing, Add Diphenydramine HCl Povidone, Usp Acetulan(Lanaetex-75) Wheat Germ Glycerides, Mix Until Dissolved
3. Add The Water And Tris Aminomethan To The Alcohol Kettle, and Mix.

In each case, after the fluid containing the composition is prepared, it is then contacted with the absorbent pads for individual use. As discussed above, the composition can be dosed into containers of the appropriate size, either containing the selected quantity of absorbent pads, or with the pads added later. Pre-absorbing the composition onto the pads before loading of the container is also contemplated. The dosed amount, such as the 1.5 fl oz. capacity discussed above, is substantially completely absorbed, though there may be some excess quantity, though not so much as to cause over saturation of the pads. Rather the degree of absorption is sufficient to treat a given target area, without causing excess dripping or running to adjacent areas.

It will be understood that various changes in the details, materials and arrangements of parts which have been herein described and illustrated in order to explain the nature of the invention, may be made by those skilled in the art within the principle and scope of the invention as expressed in the appended claims.

1. A system for applying compositions to a target area of the skin comprising a plurality of absorbent pads, a container for holding the pads, and a fluid containing the composition absorbed by the absorbent pads located in the container, the pads being sufficiently absorbent to each substantially completely absorb a unit dose of up to 1.5 fluid ounces of the fluid therein, such that the pads in the container when handled are moist but not soaking, the dose released from the pad and applied to the target area with slight pressure when the pad is placed in contact with the target area, thereby delivering the dose of the composition while avoiding spread of the fluid to non-target areas, or dripping onto nearby surfaces.

2. The system of claim 1 wherein the fluid is a povidone-iodine solution, at from 0.1-15% by weight, preferably a 10% USP solution.

3. The system of claim 1 wherein the pads are non-woven pads.

4. The system of claim 1 wherein the pads are non-woven pads made of a blend of fibers.

5. The system of claim 1 wherein the pads are non-woven pads made from a blend of rayon and polypropylene fibers.

6. A system for applying compositions to a target area of the skin comprising a plurality of absorbent pads, a container for holding the pads, and a fluid containing the composition absorbed by the absorbent pads located in the container, the pads being sufficiently absorbent to each substantially completely absorb a unit dose of up to 1.5 fluid ounces of the fluid therein, such that the pads in the container when handled are moist but not soaking, the dose released from the pad and applied to the target area with slight pressure when the pad is placed in contact with the target area, thereby delivering the dose of the composition while avoiding spread of the fluid to non-target areas, or drippinci onto nearby surfaces, wherein the fluid contains a composition selected from the group consisting of povidone-iodine, at from 0.1-15% by weight, preferably a 10% USP, Tolnaftate, at from 0.1-15% by weight, preferably 1% USP, hydrocortisone, at from 0.1-15% by weight, preferably 1% USP, Diphenhydramine, at from 0.1-15% by weight, preferably 2% USP, and Clotrimazole, at from 0.1-15% by weight, preferably 1% USP.

7. The system of claim 1 wherein the fluid is selected from the group consisting of stain producing topical agents, antihistamines, antifungal agents and antipruritic agents.

8. The system of claim 1 wherein the container is a jar, containing from 10-50 pads.

9. A method for applying a composition to a target area of the skin comprising providing a plurality of absorbent pads, providing a container for holding the pads, absorbing a fluid containing the composition onto the pads, placing the absorbent pads into the container, the pads being sufficiently absorbent for each pad to substantially completely absorb a unit dose of up to about 1.5 fluid ounces per pad of the fluid therein, removing at least one individual pad from the container, contacting the target area with slight pressure, and pressing the pad to release the unit dose of the fluid such that the unit dose of fluid is applied to the target area, thereby delivering the unit dose of fluid while avoiding spreading of the fluid to non-target areas, or dripping onto nearby surfaces.

10. The method of claim 9 wherein the fluid is povidone-iodine solution, at from 0.1-15% by weight, preferably a 10% USP solution.

11. The method of claim 9 wherein the pads are non-woven pads.

12. The method of claim 9 wherein the pads are non-woven pads made of a blend of fibers.

13. The method of claim 9 wherein the pads are non-woven pads made from a blend of rayon and polypropylene fibers.

14. The method of claim 9 wherein the fluid contains a composition selected from the group consisting of povidone-iodine, at from 0.1-15% by weight, preferably a 10% USP, Tolnaftate, at from 0.1-15% by weight, preferably 1% USP, hydrocortisone, at from 0.1-15% by weight, preferably 1% USP, Diphenhydramine, at from 0.1-15% by weight, preferably 2% USP, and Clotrimazole, at from 0.1-15% by weight, preferably 1% USP.

15. The method of claim 9 wherein the fluid is selected from the group consisting of stain producing topical agents, antihistamines, antifungal agents and antipruritic agents.

16. The method of claim 9 wherein the container is a jar, containing from 10-50 pads.
